(19) United States
(12) Patent Application Publication
(10) Pub. No.: US 2021/0369998 A1
(43) Pub. Date: Dec. 2, 2021

(54) CANNULA PLUGGING DEVICE FOR REHABILITATION AFTER TRACHEOTOMY

(71) Applicants: Xuanwu Hospital Capital Medical University, Beijing (CN); Beijing Municipal Geriatric Medical Research Center, Beijing (CN)

(72) Inventors: Junwei HAO, Beijing (CN); Haijie LIU, Beijing (CN); Xiyue ZHANG, Beijing (CN)

(73) Assignees: Xuanwu Hospital Capital Medical University, Beijing (CN); Beijing Municipal Geriatric Medical Research Center, Beijing (CN)

(21) Appl. No.: 17/333,036

(22) Filed: May 28, 2021

(30) Foreign Application Priority Data
May 28, 2020 (CN) 202020934010.3

(51) Int. Cl. A61M 16/04

(52) U.S. Cl. CPC A61M 16/0445; A61M 16/0465; A61M 16/0488

(57) ABSTRACT

A cannula plugging device for rehabilitation after tracheotomy relates to the field of medical devices. The cannula plugging device includes a plugging device bracket. An outer wall of the plugging device bracket is fitted with a ring-shaped outer airbag. An inner wall of the plugging device bracket is fitted with a ring-shaped inner airbag. Two inflation tubes connecting the outer airbag and the inner airbag respectively are fitted on the plugging device bracket. The present invention makes the outer airbag and the inner wall of tracheotomy cannula pressed together by inflating the outer airbag. By gradually inflating the inner airbag and adjusting the size of the inner diameter of the lumen, the opening of the trachea gradually changes, so that the patient can gradually adapt to the process of plugging the cannula.

### CROSS-REFERENCE TO RELATED APPLICATION

This application claims the priority benefit of China application serial no. 202020934010.3, filed on May 28, 2020. The entirety of the above-mentioned patent application is hereby incorporated by reference herein and made a part of this specification.

### BACKGROUND

### Technical Field

The invention relates to the medical device field, and specifically relates to a cannula plugging device for rehabilitation after tracheotomy.

### Description of Related Art

Tracheotomy is a common emergency surgery to establish an artificial airway for the symptoms of pharyngeal obstruction and lower airway secretion retention due to various disease causes (for example, impaired consciousness, loss of cough reflex, respiratory muscle paralysis, trauma, surgery, respiratory disease). When the disease is cured, after tube plugging tests, the tube can be removed only after making sure that the patient is adaptable to the normal physiological airway.

For some patients with severe stroke and myasthenia gravis, their airways are left in the incised state for too long to rapidly accomplish tube plugging tests, because when the airway is directly connected to extracorporeal air, the respiratory void lumen is reduced and the breathing resistance is lower, and the patients have adapted to a less resistant non-physiological airway with an open airway. Therefore, there is a need for an accessible pre-extubation respiratory rehabilitation device. But currently the tracheotomy cannulas used in the clinic lack a matching plugging device, physicians and nurses have to use self-made pipe plugging devices, such as corks, swabs, gloves, dressings, vacuum caps, Murphy drippers, syringe plungers, and other plugging methods to plug the tube, to plug the pipe. And for patients who are tracheotomized with respiratory function, there is currently no specific respiratory function training device.

The self-made cannula plugging devices have the following major problems. First, the crafting processes of the self-made devices are not convenient, and because of the dimensional difference, it can be time-consuming and labor-intensive to make a proper device, for example, the wrapping tape step when using the existing self-made vacuum cap wrapped with tape for plugging. Second, the self-made cannula plugging devices are difficult to achieve aseptic requirements, and easy to get contaminated, for example, the existing self-made wooden plugs for blocking, not only difficult to disinfect and sterilize, but also easy to adhere to the sputum after long time use. Third, the self-made plugging devices are fixed to the cannulas relied mainly on their own calibers matching the cannula calibers to increase the friction between them, are not suitable for cannulas of different diameters, and easy to cause patient discomfort when being mounted to the cannulas. Such as the patent CN208448358U which relies on caliber matching principle to ensure airtightness and fastness between the plugging device and the cannula, but inconvenient to mount. Fourth, the self-made plugging devices cannot perform half plugging of cannulas simply and conveniently. The common plugging devices that can perform half plugging of cannulas are all with paddles structure, which are at risk of being accidentally inhaled by patients because the parts on the cannula plugging device are easy to break and fall off when the cannulas are in use. Such as the patent publication of CN201631858U of which the fan-shaped panels are easy to fall off after long time use. Fifth, the airtightness of the self-made plugging devices is generally poor. Such as the patent publication of CN208003225U, the top end of the plugging device is a louvered structure, which is prone to air leakage makes it impossible to accurately assess the total blockage of the tube cannulas. Sixth, the structures of the self-made plugging device are fixed, so they cannot adapt to the different diameters of the tracheotomy cannulas. Such as the patent publication of CN203017535U which needs to designs different types of plugging devices, making clinical use extremely inconvenient.

Therefore, there is an urgent need for a plugging device that is clean, resistant to contamination, easy to operate, safe, and can plug the tracheotomy cannulas progressively.

### SUMMARY

The present invention provides a cannula plugging device for rehabilitation after tracheotomy, to meet the clinical needs of tracheotomy patients in plugging the cannula tube, and help the tracheotomy patients to recover better, enables them to transition from tracheal ventilation to breathing through the mouth and nose smoothly, and achieve functional reconstruction. Also, the invention is more solid and clean after installation, easy to operate, more convenient and safe when installed.

To achieve the object, the cannula plugging device of the present invention includes a plugging device bracket. An outer wall of the plugging device bracket is fitted with a ring-shaped outer airbag, and an inner wall of the plugging device bracket is fitted with a ring-shaped inner airbag. Two inflation tubes connecting the outer airbag and the inner airbag respectively are fitted on the plugging device bracket.

Further regarding the cannula plugging device, the plugging device bracket is cannula shape.

Further regarding the cannula plugging device, the plugging device bracket has an outward turning brim at one end.

Further regarding the cannula plugging device, the plugging device bracket is integrated with the outward turning brim.

Further regarding the cannula plugging device, the plugging device bracket is of rigid structure.

Further regarding the cannula plugging device, the outer airbag is at least one in number, and the multiple outer airbags are spaced along an axis of the plugging device bracket.

Further regarding the cannula plugging device, each of the inflation tubes is mounted with an air valve.

It is an object of the present invention to provide a cannula plugging device which has a small and lightweight structure, which makes it easy for patients to move around after it being mounted on the tracheotomy cannula and it is easy to mount, so that the mounting process does not cause discomfort to patient.

It is another object of the present invention to provide a cannula plugging device which makes the outer airbag pressed against the inner wall of the tracheotomy cannula by inflating the outer airbag, thus making the present invention more firmly installed. Also, by gradually inflating the inner balloon to adjust the size of the inner diameter of the lumen, makes the opening angle of the patient's trachea gradually changes so that the patient can gradually adapt to the plugging process which is conducive to the reconstruction of the patient's respiratory function.

It is further object of the present invention to provide a cannula plugging device which can be mounted on tracheotomy cannula of different internal diameters by inflating the outer air bag, thus, it is suitable for different sizes of tracheotomy cannula and has a wider range of application.

It is further object of the present invention to provide a cannula plugging device which when using, the inner air bags and the outer air bags can be inflated or deflated by using a common syringe to fill or draw air into the inflatable tubes, is convenient to use and suitable for the rehabilitation training of breathing function before plugging and extubation.

An even further object of the present invention is to provide a cannula plugging device which when using, due to the smooth surface of the inner air bags and the outer air bags, prevents sputum from adhering to them and makes it easy to clean them, so that, microorganisms such as bacteria less likely to grow.

Lastly, it is an object of the present invention to provide a cannula plugging device which sets plugging device bracket integrated with the outward turning brim to prevent the danger caused by small parts falling into the airway making it safer when using.

To make the aforementioned more comprehensible, several embodiments accompanied with drawings are described in detail as follows.

### BRIEF DESCRIPTION OF THE DRAWINGS

The following is a brief description of the annexed drawings to be used in specific embodiments in order to better understand the present invention. It is obvious that the drawings in the following description are some embodiments of the present invention and represent schematics, not physical drawings, and cannot be regarded as a limitation of the present patent, and for those of ordinary skill in the art, other drawings obtained without creative labor according to the drawings of the present invention are within the scope of the present invention.

FIG. 1 is a structure drawing of a cannula plugging device for rehabilitation after tracheotomy of the preferred embodiment.

FIG. 2 is an assembled structure drawing of a progressive cannula plugging device of the preferred embodiment.

FIG. 3 is a working flowchart of the progressive cannula plugging device of the preferred embodiment.

### DESCRIPTION OF THE EMBODIMENTS

With reference to the drawings, the preferred embodiments will provide further details of the present invention.

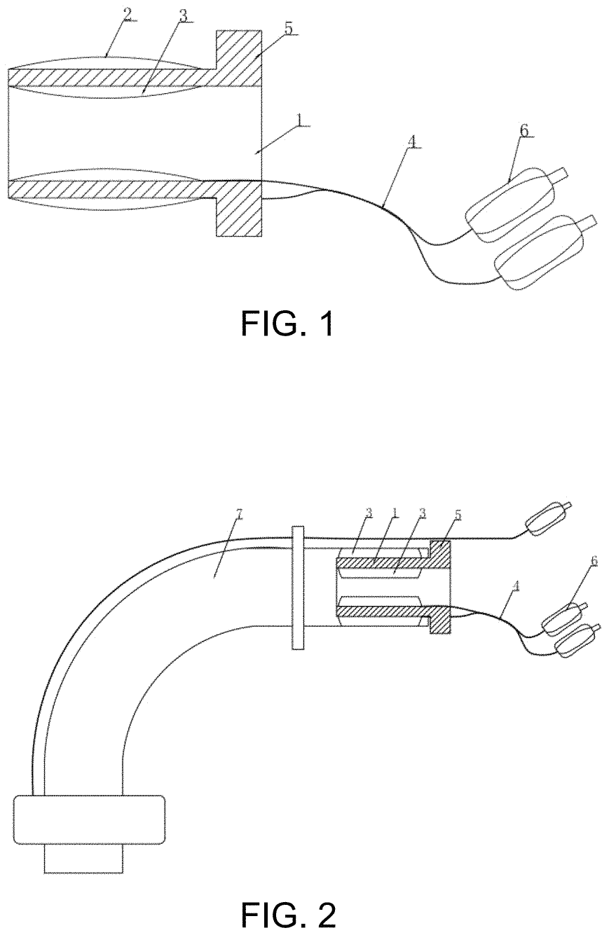

FIG. 1 and FIG. 2 show a cannula plugging device for rehabilitation after tracheotomy provided by the present invention, provided by the present invention. The cannula plugging device includes a plugging device bracket 1. An outer wall of the plugging device bracket 1 is fitted with a ring-shaped outer airbag 2, and an inner wall of the plugging device bracket is fitted with a ring-shaped inner airbag 3. Two inflation tubes 4 connecting the outer airbag 2 and the inner airbag 3 respectively are fitted on the plugging device bracket 1.

The outer diameter of the plugging device bracket 1 is smaller than that of the tracheotomy cannula 7, so that the plugging device bracket 1 can be inserted in the tracheotomy cannula 7 smoothly. The outer airbag 2 is fixed to the outer surface of the plugging device bracket 1, and the outer airbag 2 is ring-shaped, so that after the outer airbag 2 is fully inflated and bulged, form an ring-shaped sealed structure, which not only prevents the plugging device bracket 1 from falling off, but also effectively prevents the plugging failure caused by the gap between the outer surface of the plugging device bracket 1 and the tracheotomy cannula 7.

The inner airbag 3 is fixed to the inner surface of the plugging device bracket 1, and the inner airbag 3 is ring-shaped. When the inner airbag 3 is fully inflated and bulged, the inner airbag 3 can completely seal the inside of plugging device bracket 1.

The two inflatable tubes 4 respectively connecting the outer airbag 2 and the inner airbag 3 are respectively fixed on the inner surface of the plugging device bracket 1 and the outer surface of plugging device bracket 1, so that when the outer airbag 2 or the inner airbag 3 needs to be inflated and bulged, the outer airbag 2 or the inner airbag 3 can be inflated through the corresponding inflatable tube 4. In order to prevent the inflatable tube 4 from tangling, the middle of the inflatable tubes 4 can be set side-by-side fixed.

When the outer airbag 2 is inflated and gradually bulged, the inflated and bulged outer airbag 2 is pressed against the inner wall of the tracheotomy cannula 7, not only make the plugging device bracket 1 fixed with the tracheotomy cannula 7, but also form a sealing structure between the outer wall of the plugging device bracket 1 and the inner wall of the tracheotomy cannula 7. By controlling the amount of air inside the inner air bag 3, the bulge degree of the inner air bag varies, so that various sizes of ventilation inner diameter can be obtained.

The plugging device bracket is cannula shape. After the plugging device bracket 1 is fixed to the tracheotomy cannula 7, the cannula shape of the plugging device bracket 1, allows the tracheotomy cannula 7 remain connected to the outside world through the plugging device bracket 1 when the inner airbag 3 is not fully inflated and bulged, and make sure that the sealing structure is formed between the outer surface of the plugging bracket 1 and the inner surface of the tracheotomy cannula 7 after the outer airbag 2 is fully bulged, so as to prevent the failure of the cannula plugging due to the gap between the outer surface of the plugging device bracket 1 and the inner surface of the tracheotomy cannula 7 when the patient needs complete cannula plugging.

The plugging device bracket 1 has an outward turning brim 5 at one end. The diameter of the outward turning brim 5 is larger than the diameter of the plugging device bracket 1, thus can not only avoid the plugging device bracket 1 falling directly into the tracheotomy cannula 7 when using, but also makes the removal of the plugging device bracket 1 from the tracheotomy cannula 7 easier, after the plugging device bracket 1 is mounted into the tracheotomy cannula 7.

The plugging device bracket is integrated with the outward turning brim. When producing the plugging device bracket 1, the outward turning brim 5 and the plugging device bracket 1 can be injection molded together directly, thus not only makes the production of the plugging device bracket 1 more convenient, but also makes the structure of the plugging device bracket 1 and the outward turning brim 5 more solid, ensures the quality of the product of the invention, effectively prevents the danger caused by small parts falling off into the airway, and enhances the security performance.

The plugging device bracket is of rigid structure, thus effectively prevent the outer airbag 2 or inner airbag 3 from deforming the plugging device bracket 1 due to the extrusion of the outer airbag 2 or inner airbag 3 after inflation, so that the outer airbag 2 can be pressed between the outer surface of the plugging device bracket 1 and the tracheotomy cannula 7 after being inflated, to ensure that the gap between the cannula plugging device and the tracheotomy cannula 7 can be sealed.

The outer airbag is at least one in number. The multiple outer airbags 2 are spaced along the axis of the plugging device bracket, and the gap between two adjacent outer airbags 2 can be equal or unequal. And when the outer airbags 2 are multiple, the multiple outer airbags 2 can be connected so that when the outer airbags 2 are inflated, one inflation tube 4 can satisfy the simultaneous inflation of multiple outer airbags 2, thus not only makes the structure simpler, but also makes it easier to inflate the outer airbags 2. By setting multiple outer airbags 2, and make them work together, it not only makes the lugging device bracket 1 fitted to the tracheotomy cannula 7 more securely, but also makes the sealing effect between the outer surface of the lugging device bracket 1 and the inner surface of the tracheotomy cannula 7 better.

The inflation tube 4 is mounted with air valve 6, and the air valve 6 positioned at the inlet end of the inflation tube 4. By installing the air valve 6, the outer airbag 2 or the inner airbag 3 can be inflated by injecting air through the air valve 6 into the inflation tube 4 with an empty needle when needed.

Both the outer airbag 2 and the inner airbag 3 in the present invention are elastic, so that the outer airbag 2 and the inner airbag 3 can be inflated many times of different degrees, ensuring the service life of the product of the present invention. At the same time, in order to facilitate the inflation of the inner airbag 3 and the outer airbag 2, the inner airbag 3 and the outer airbag 2 can be set to two different colors, and the two inflation tubes 4 and the two air valves 6 can be set to different colors, thus effectively preventing the confusion of the inflation of the inner airbag 3 or the outer airbag 2.

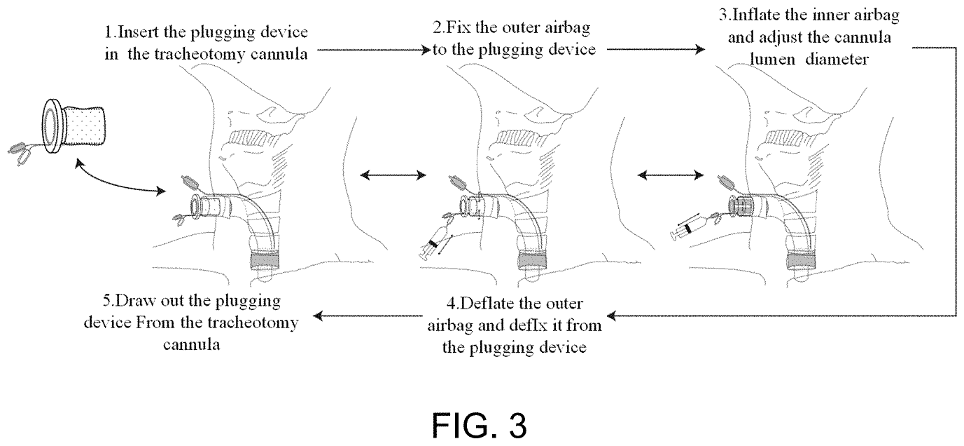

As shown in FIG. 3, when the product of the present invention needs to be fitted on the tracheotomy cannula 7, first insert the plug tube bracket 1 in the tracheotomy cannula 7, then use the empty needle to inflate the outer air bag 2 through the air valve 6 to the inflation tube 4 connected with the outer air bag 2. Air enters into the outer airbag 2 via the inflation tube 4, and makes the outer airbag 2 gradually bulge, while the outer airbag 2 is bulging, the outer airbag 2 is gradually compressed with the tracheotomy cannula 7. When the outer airbag 2 is completely bulged, the outer airbag 2 is completely compressed with the tracheotomy cannula 7, which makes the lugging device bracket 1 fixed with the tracheotomy cannula 7.

When the patient requires gradual rehabilitation and need to control the ventilation of the tracheotomy cannula 7, use the empty needle to inflate the inner air bag 3 through the air valve 6 to the inflation tube 4 connected with the inner air bag 3 to make the inner airbag 3 bulge to a certain extent. When the ventilation volume of the tracheotomy cannula 7 needs to be further controlled, the empty needle is again used to inflate the inner air bag 3 so that the inner air bag 3 bulges further, making the passage of the lugging device bracket 1 smaller and thus making the ventilation volume of the tracheotomy cannula 7 smaller. When the tracheotomy cannula 7 needs to be completely plugged, the empty needle is used again to inflate the inner balloon 3, so that the inner balloon 3 is completely bulged, thus enabling the channel of the plugging bracket 1 is plugged, and further enabling the tracheotomy cannula 7 to be plugged.

When the patient has a generally good condition, the pharyngeal muscles are coordinated, and the clinical assessment is suitable for one-time plugging, the internal airbag 3 can be inflated to the maximum inflation volume at one time, that is, the tracheotomy cannula 7 can be fully plugged, thus making the cannula plugging process more convenient and faster.

When using the product of the present invention, if the patient has breathing difficulty, the air of the inner air bag 3 can be withdrawn first to increase the ventilation diameter between the plug tubes, so that the ventilation diameter of the tracheotomy cannula 7 becomes larger. If the patient still cannot breathe smoothly, the air of the outer air bag 2 can be withdrawn, and then the product of the present invention can be removed from the tracheotomy cannula 7, so that the airway obstruction can be released quickly.

The product of the present invention has a lightweight structure, which is convenient for the patient to move around after inserted in the tracheotomy cannula 7. And it is easy to mount, so that the mounting process will not cause discomfort to the patient. By inflating the outer airbag 2, the outer airbag 2 is pressed against the inner wall of the tracheotomy cannula 7, so that the product is installed more securely. And by gradually inflating the inner airbag 3, the opening degree of the trachea gradually changes, so that the patient can gradually adapt to it, which is conducive to the reconstruction of the patient's respiratory function.

The product of the present invention can be mounted on tracheotomy cannula 7 of different inner diameters, thus applicable to tracheotomy cannula 7 of different sizes, with a wider scope of application. In addition, the inflation and deflation of the inner airbag 3 and the outer airbag 2 can be operated directly by ordinary syringes, which is convenient to use for the rehabilitation training of breathing function before plugging and extubation

Since the smooth surfaces of the inner airbag 3 and outer airbag 2 prevent sputum from adhering to them and make it easy to clean them, thus, microorganisms such as bacteria less likely to grow.

In particular, it should be clear that the above description is only an exemplary description, not all embodiments, and not a limitation of the way the present invention is implemented, and other different forms of variations or changes can be made based on the above description for those skilled in the art. Any modification, equivalent substitution and improvement, etc. made within the concept and principles of the present invention shall be included within the scope of the present invention.

What is claimed is:

1. A cannula plugging device for rehabilitation after tracheotomy, the cannula plugging device comprising:
a plugging device bracket, wherein an outer wall of the plugging device bracket is fitted with a ring-shaped outer airbag, an inner wall of the plugging device bracket is fitted with a ring-shaped inner airbag, two inflation tubes connecting the outer airbag and the inner airbag respectively are fitted on the plugging device bracket.

2. The cannula plugging device of claim 1, wherein the plugging device bracket is cannula shape.

3. The cannula plugging device of claim 1, wherein the plugging device bracket has an outward turning brim at one end.

4. The cannula plugging device of claim 3, wherein the plugging device bracket is integrated with the outward turning brim.

5. The cannula plugging device of claim 1, wherein the plugging device bracket is of rigid structure.

6. The cannula plugging device of claim 1, wherein the outer airbag is at least one in number, the multiple outer airbags are spaced along an axis of the plugging device bracket.

7. The cannula plugging device of claim 1, wherein each of the inflation tubes is mounted with an air valve.
